(19) United States
(12) Patent Application Publication
(10) Pub. No.: US 2024/0076803 A1
(43) Pub. Date: Mar. 7, 2024

(54) Method for Library Preparation in Next Generation Sequencing by Enzymatic DNA Fragmentation

(71) Applicant: Miltenyi Biotec B.V. & Co. KG, Bergisch Gladbach (DE)

(72) Inventor: Matthias Wahl, Bergisch Gladbach (DE)

(21) Appl. No.: 18/274,541

(22) PCT Filed: Jan. 28, 2022
(86) PCT No.: PCT/EP2022/051979

§ 371 Date: Jul. 27, 2023

(30) Foreign Application Priority Data
Jan. 29, 2021 (EP) 21154220.4

(51) Int. Cl. C40B 50/06

(52) U.S. Cl. CPC C40B 50/06

(57) ABSTRACT

The invention is directed to a method for obtaining a nucleic acid library of a sample comprising polynucleotides comprising the steps a. multiplying the polynucleotides by a polymerase b. fragmenting the multiplied polynucleotides by creating nicks c. coupling an oligonucleotide sequence to the nicks to create the target library wherein step a) is performed by providing A, T, G, C and U nucleotides wherein the molar ratio of T and U is between 200:1 and 5:1 and step b) is performed by excision of the U nucleotides. characterized in that after step b), the nicks are provided with a polymerase exhibiting 5′ #3′ exonuclease activity, thereby filling in the 3′ recessing ends and removing the 5′ overhangs of the nicks.

### BACKGROUND

Next Generation Sequencing is an emerging technology extending to all areas of Biomedical Research and Clinical Diagnostics. One of the key steps in Next Generation Sequencing is the Library Preparation (Library Prep).

During Library Prep, the DNA to be sequenced is provided with specific sequences on both ends (adaptor sequences), to which the sequencing primer or amplification primers bind. To these adaptor sequences further sequences providing other information may be added, like specific sequences (barcodes) for the assignment of a Next Generation Sequencing read to a particular sample or a cell or a molecule.

Many Next Generation Sequencing assays require that the DNA of interest is being fragmented. Fragmentation techniques are for example disclosed in:
Hess J F, Kohl T A, Kotrová M, Rönsch K, Paprotka T, Mohr V, Hutzenlaub T, Brüggemann M, Zengerle R, Niemann S, Paust N. Library preparation for next generation sequencing: A review of automation strategies. Biotechnol Adv. 2020 Jul-Aug; 41:107537. doi: 10.1016/j.biotechadv.2020.107537. Epub 2020 Mar 19. PMID: 32199980.
Head S R, Komori H K, LaMere S A, et al. Library construction for next-generation sequencing: overviews and challenges. Biotechniques. 2014; 56(2):61-passim. Published 2014 Feb. 1. doi:10.2144/000114133

Further information concerning sequencing techniques involving fragmentation can be found in WO2010/148039, WO2016/114970 and WO2015/200541 and in the reference list cited below.

The known fragmentation techniques include:
Physical shearing of nucleic acids, for example using the Covaris ultra-sonicator.
Enzymatical fragmentation of nucleic acids using nucleases.
Fragmentation of nucleic acids with the use of transposase, which semi-randomly inserts adaptor sequences into DNA.

Physical shearing requires the purchase of an expensive instrument, cannot be automated, and cannot be parallelized for multiple samples when using a single instrument.

The enzymatic fragmentation and tagmentation procedures have a significant disadvantage: The degree of fragmentation and tagmentation is very sensitive towards time and the input amount (input DNA), therefore this step has to be very tightly controlled (by accurate quantification of input amount and incubation time), and reagents have to be pre-chilled in order to avoid that the reaction starts prematurely.

The requirement that reactions have to be pipetted on ice provides a significant usability constraint, since many laboratories are not equipped with equipment for chilling reagents (especially diagnostics labs using multiple physically separated rooms for contamination prevention refrain from using equipment for chilling reagents like ice machines). Also, incubations at low temperature make automation of the pipetting steps challenging because not every automation solution is capable of cooling reagents.

The time criticality of incubation steps also impacts the scalability of workflows as some of the reactions will start immediately after addition of the sample.

The methods of the prior art are compared in the following table

| | | | Enzymatic |
|---|---|---|---|
| | | | fragmentation using |
| | Shearing | Tagmentation | nucleases |
| Sequence | Low | Sequence bias: strong | Lower sequence bias |
| bias | | bias in low GC regions | compared to |
| | | for Illumina Nexteraq | Tagmentation |
| | | kits using mutated Tn5 | |
| | | transposase | |
| Importance | | High | High |
| of accurate | | | |
| quantification | | | |
| Importance | | High | High |
| of controlled | | | |
| time and | | | |
| temperature | | | |
| Multistep | Yes: End repair and | Yes | Very difficult: |
| single-tube | Ligation can be | | majority of workflows |
| workflows | conducted in single tubes | | require multiple clean- |
| possible? | | | up steps |
| Automated | No-shearing has to be | Difficult: requires | Difficult: requires |
| workflows | conducted manually | accurate quantification | accurate quantification |
| possible? | | of low conc. DNA; | of low conc. DNA; |
| | | requires time- and | requires time- and |
| | | temperature controlled | temperature controlled |
| | | pipetting | pipetting |

In order to avoid the downsides of the known methods, it is proposed to use dUTP (deoxyuridine triphosphate) and enzymes catalyzing the excision of uracil nucleotides for fragmenting DNA during the Library Prep workflow of a Next Generation Sequencing assays.

### SUMMARY

It was therefore an object of the invention to provide a method for obtaining a nucleic acid library of a sample comprising polynucleotides comprising the steps
a. multiplying the polynucleotides by a polymerase
b. fragmenting the multiplied polynucleotides by creating nicks
c. coupling an oligonucleotide sequence to the nicks to create the target library wherein step a) is performed by providing A, T, G, C and U nucleotides wherein the molar ratio of T and U is between 200:1 and 5:1, preferable between 150:1 and 25:1, more preferable between 50:1 and 5:1 and step b) is performed by excision of the U nucleotides. characterized in that after step b), the nicks are provided with a polymerase exhibiting 5′ 4 3′ exonuclease activity, thereby filling in the 3′ recessing ends and removing the 5′ overhangs of the nicks.

As A, T, G, C and U nucleotides, the known building blocks for oligonucleotide synthesis like dUTP nucleotides can be used.

Preferable, the A, T, G, C and U nucleotides are provided as Adenosine 5′-Triphosphate (ATP), 2′-Deoxyadenosine 5′-Triphosphate (dATP), Thymidine 5′-Triphosphate (TTP), 2′-Deoxythymidine 5′-Triphosphate (dTTP), Guano sine 5′-Triphosphate (GTP), 2′-Deoxyguanosine 5′-Triphosphate (dGTP), Cytidine 5′-Triphosphate (GTP) and 2′-Deoxycytidine 5′-Triphosphate (dGTP), 2′-Deoxyuridine, 5′-Triphosphate (dUTP) or Uridine-5′-triphosphate (UTP). The person skilled in the art is aware that these compounds are available as natural occurring form or chemically modified as derivative. In the method of the invention, the natural occurring form of the nucleotides and/or a derivative thereof (i.e. chemically modified version can be used.

The target nucleic acid library obtained by method of the invention may be sequenced. The method for sequencing is not particular important and any method for sequencing known in the art can be used for this purpose.

The oligonucleotide sequence coupled to the nicks is preferable an adaptor or primer sequence like a PCR starter sequence which can be used for amplification purposes, or a sequencing primer binding sequence which can be used for sequencing the target nucleic acid library.

### BRIEF DESCRIPTION OF THE DRAWINGS

FIG. 1 shows the principle of the method of the invention in a generic process.

FIG. 2 depicts a variant using messenger RNA as starting material

FIG. 3 depicts a variant using targeted enrichment (specific amplification) of one or multiple nucleic acid targets

FIG. 4 depicts a variant using linear amplification for the amplification of nucleic acids in the presence of dUTP nucleotides with Phi29 polymerase

FIG. 5 depicts a method for generating a nucleic acid library using nucleic acids fragmented with the method of invention by generating blunt ends followed by ligation of a specific nucleotide adapter.

FIG. 6 depicts multiple different adaptor designs that can be used for the method shown in FIG. 5.

FIG. 7 shows a variant of the method wherein the nucleic acid fragments are first denatured to obtain single-stranded nucleic acid fragments which are then provided with a specific nucleotide adapter.

FIG. 8 shows a variant of the method wherein the nucleic acid fragments are first denatured to obtain single-stranded nucleic acid fragments which are then ligated with poly-A tails at the 3′ ends

FIGS. 9 to 12, 14 and 16 to 18 show experimental results

FIGS. 13 and 15 summarizes the differences between the method of the invention and the prior art.

### DETAILED DESCRIPTION

The method of the invention provides a novel approach for statistical fragmenting of polynucleotides that can be utilized for the generation of sequencing libraries derived from target nucleic acids.

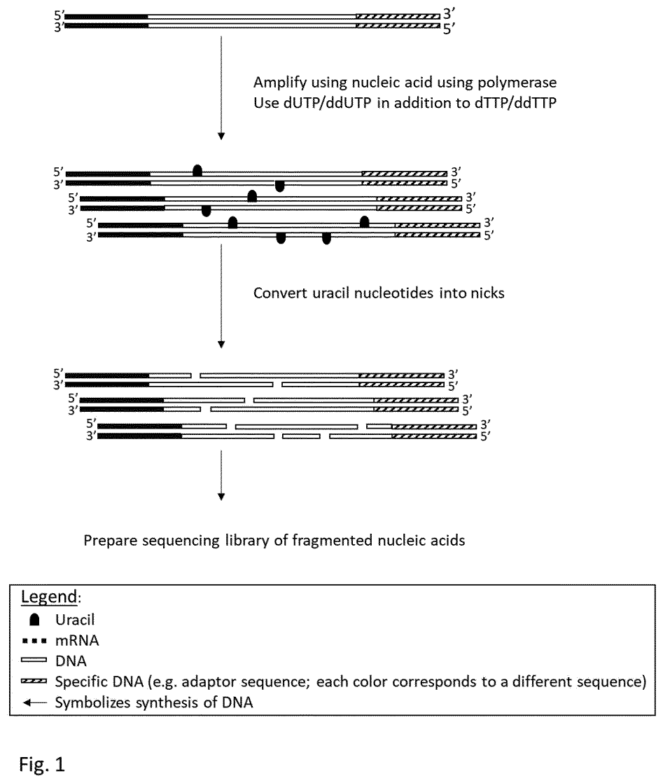

Instead of methods of prior art which use physical shearing, nucleases or transposases for statistical fragmentation of target nucleic acids, this method incorporates uracil nucleotides during a polymerisation step which subsequently are converted into nicks (FIG. 1).

A key step of the method is the initial polymerisation step which is already part of many nucleic acid library preparation methods. During this polymerisation step, dUTP or ddUTP nucleotides are incorporated into the polynucleotides being synthesized.

In the method of the invention, the target nucleic acids may be derived from genomic DNA, RNA or a plurality of DNA molecules comprising 50 to 2000 nucleotides.

The method according to invention provides a robust pathway for statistical fragmenting of polynucleotides so that that at least one of the steps a) b) or c) is performed without purification of the obtained (intermediate) product.

Step a—Multiplying the Target Nucleic Acids

Preferable before step a, the target nucleic acids are provided at the 3′ and 5′ ends with primer sequences for amplification.

Multiple polymerase-based methods exist which can be used for incorporating uracil bases into nucleic acid. The following sections contain three different methods that already are part of many workflows for the generation of sequencing libraries:
Incorporation of uracil nucleotides during cDNA synthesis
Incorporation of uracil nucleotides during PCR amplification
Incorporation of uracil nucleotides during linear amplification

Incorporation of Uracil Bases During cDNA Synthesis:

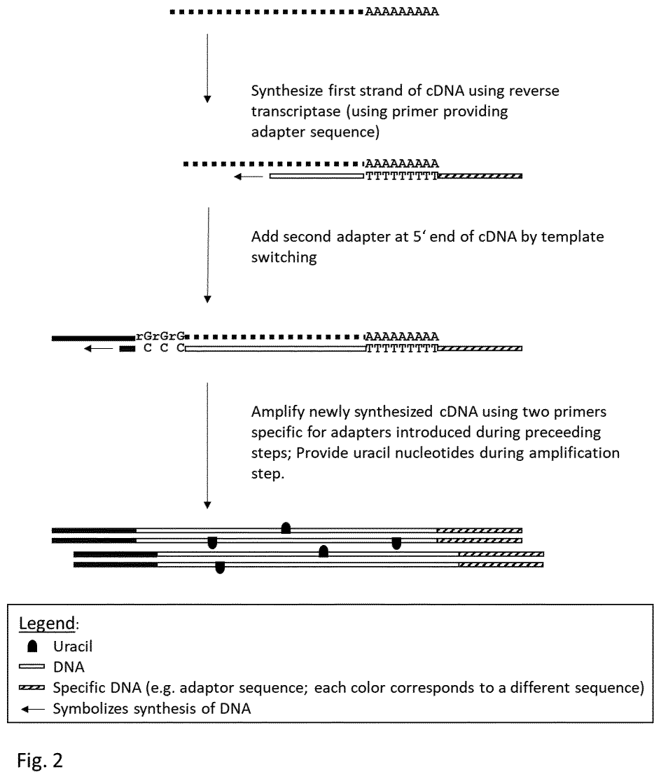

FIG. 2 depicts a method using messenger RNA as starting material. First, cDNA is synthesized using reverse transcriptase and an oligo(dT) primer (oligonucleotide with multiple T nucleotides); the oligo(dT) primer may contain one or more additional nucleotides at the 3′ end; the oligo(dT) primer may also contains a specific nucleic acid sequence 5′ to the oligo(dT) stretch (adaptor 1 containing a specific primer binding sequence 1; this adaptor is depicted with upward diagonal stripes).

Once the reverse transcriptase reaches the 5′ end of the mRNA, a second specific sequence is introduced (adaptor 2 containing specific primer binding sequence 2; this adaptor is depicted with a solid box) using the template switching approach (Chenchik et al., 1998).

The two specific primers may also be introduced using random priming during reverse transcription and/or during a subsequent second strand cDNA synthesis step.

This newly synthesized cDNA is then amplified in the presence of dUTP by a polymerase using primers specific to the primers incorporated during the cDNA synthesis.

Alternatively, UTP or dUTP nucleotides may already be added during the reverse transcription and/or second strand synthesis step; in this case, the amplification step may be omitted.

Incorporation of Uracil Bases During PCR Amplification:

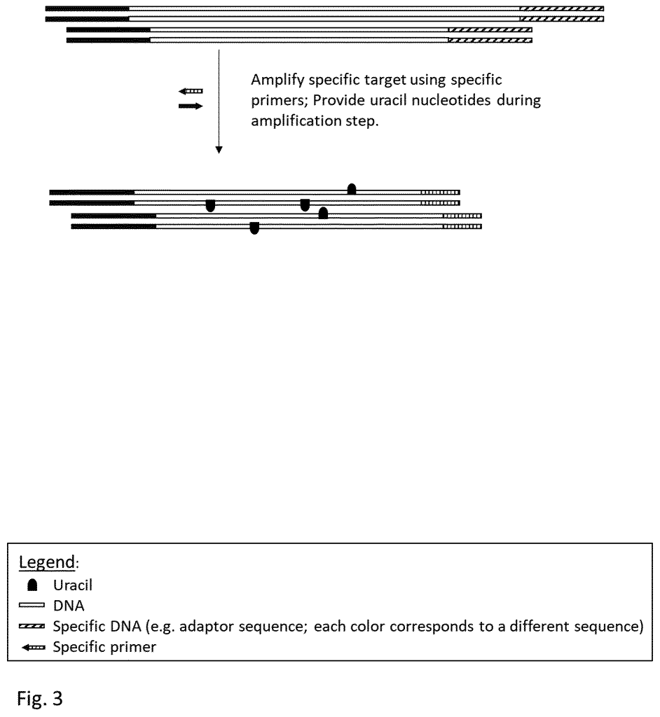

Preferable, step a) is conducted by polymerase chain reaction. FIG. 3 depicts an method using targeted enrichment (specific amplification) of one or multiple nucleic acid targets; in this example, the target enrichment is conducted by using one primer specific to a sequence already present in the template nucleic acid, and a second primer specific to the target or targets of interest. The targeted amplification is conducted using specific primers, a polymerase and nucleotides, including dUTPs.

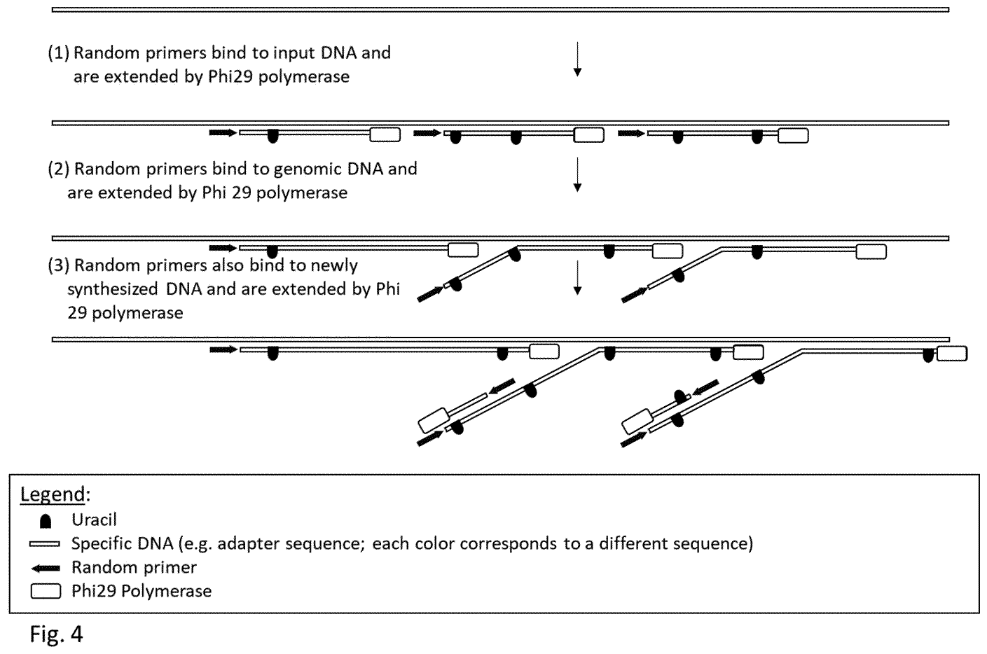

The amplification steps mentioned in the descriptions for FIG. 3 and FIG. 4 can be conducted by polymerase chain reaction using Taq polymerase (thermostable DNA polymerase I of Thermus aquaticus) or other proof-reading polymerases capable of mediating polymerase chain reactions. Alternatively, the amplification can be achieved using Loop-mediated isothermal amplification.

Incorporation of Uracil Bases During Linear Amplification:

FIG. 4 depicts a method using linear amplification for the amplification of nucleic acids in the presence of dUTP nucleotides, for example using Phi29 polymerase for the amplification of whole genomes (Silander et al., 2008).

Step b—Fragmentation of the Polynucleotides

The newly synthesized nucleic acids are subsequently treated with an enzyme mixture capable of removing uracil nucleotides thereby creating nicks.

Preferable, nicks are generated by a providing one or more enzymes selected from the group consisting of DNA glycosylases (for example Uracil DNA Glycosylase), endonucleases (for example Endonuclease III or Endonuclease VIII), or engineered recombinant proteins (for example USER enzyme) and thermolabile engineered recombinant proteins (for example USER II enzyme).

The use of USER enzyme and thermolabile USER II enzyme is exemplary for any recombinant protein and the term USER hereinafter shall be interpreted for “recombinant protein”.

Examples for such enzyme mixtures are uracil-DNA glycosylase (UDG) and endonuclease III or UDG and endonuclease VIII (Melamade et al, 1994; Jiang et al, 1007). Alternatively, commercial enzymes or enzyme mixes like the USER enzyme or the thermoliable USER enzyme from New England Biolabs may be used (Cat. No M5508 and M5507, New England Biolabs, Ipswich, MA, USA).

In attrition to providing enzymes, the creation of nick can be performed be applying elevated temperatures of chemicals.

The number of uracil bases in the newly synthesized nucleic acids can be tuned by adjusting the ratio between dUTP/ddUTP and dTTP/ddTTP nucleotides during the polymerisation step. The higher the relative abundance of dUTP/ddUTP, the more uracil nucleotides will be incorporated (replacing thymidine nucleotides).

Since nicks are specifically generated at the sites of uracil nucleotides, the fragment length is proportional to the relative abundance of dUTP/ddUTP during the polymerization step. Therefore, the fragment length can be statistically tuned by adjusting the relative abundance of dUTP/ddUTP in the polymerization step.

Step c—Coupling Oligonucleotides to the Nicks

This section lists multiple preferred embodiments for creating nucleic acid libraries from nucleic acid fragments generated by incorporation of uracil nucleotides and subsequent excision of these uracil nucleotides.

Optionally, the oligonucleotide sequences coupled to the nicks are primer sequences.

To exemplify these embodiments, nucleic acid fragments generated using the method introduced in FIG. 2 (mRNA converted to cDNA with 5′ and 3′ specific adaptors introduced using template switching and oligo(dT) priming, respectively) are depicted.

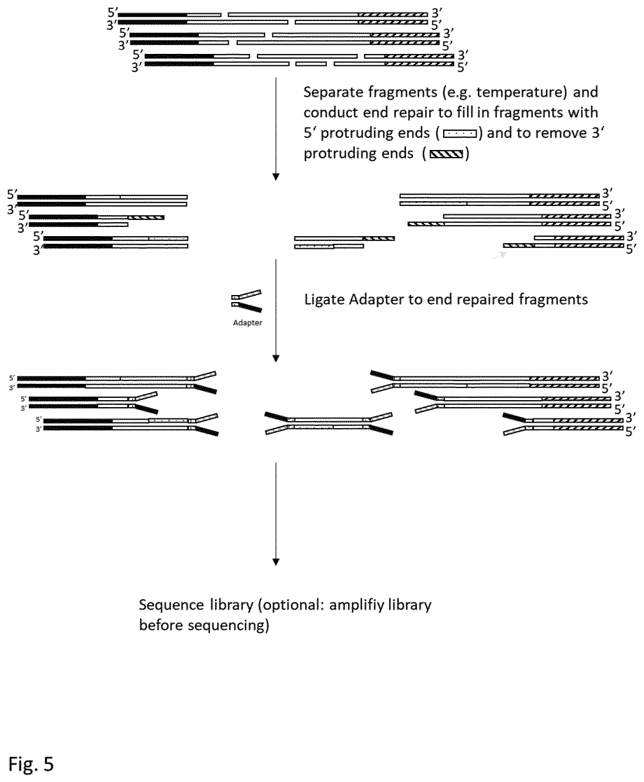

The embodiment shown in FIG. 5 first creates blunt ends to which a specific oligonucleotide adaptor is subsequently ligated. This is achieved by separating the fragmented nucleic acids followed by the treatment of the fragmented nucleic acids with an enzyme or an enzyme mix exhibiting a 5′→3′ polymerase activity and a 3′→5′ exonuclease activity.

At fragments with 5′ protruding ends, a reverse complimentary second strand is being synthesized using the 5′→3′ polymerase activity (“fill-in”); at fragments with 3′ protruding ends, the protrusion is removed using the 3′→5′ exonuclease activity. After this treatment, all fragments have blunt ends.

In a modification of this embodiment, one or more A nucleotides are added to the 3′ end of the fragments (“A-tailing”). This A-tailing is achieved by either using an enzyme with A-tailing activity for the reaction above, or by an additional treatment with an enzyme exhibiting A-tailing activity.

Next, a double-stranded oligonucleotide (adaptor) is ligated to the fragments (either through blunt end ligation or with a double stranded oligonucleotide containing a T overhang in case the fragments were treated with an enzyme with A-tailing activity). The double-stranded adaptor used for ligation contains one or two specific primer binding sequences. In a modification of this embodiment, the adapter might be partially single-stranded.

In a variant of the invention, the nucleic acid library may be sequenced. For this purpose, the primer sequence/these primer sequences added during adapter ligation can be used for subsequent sequencing of the nucleic acid library.

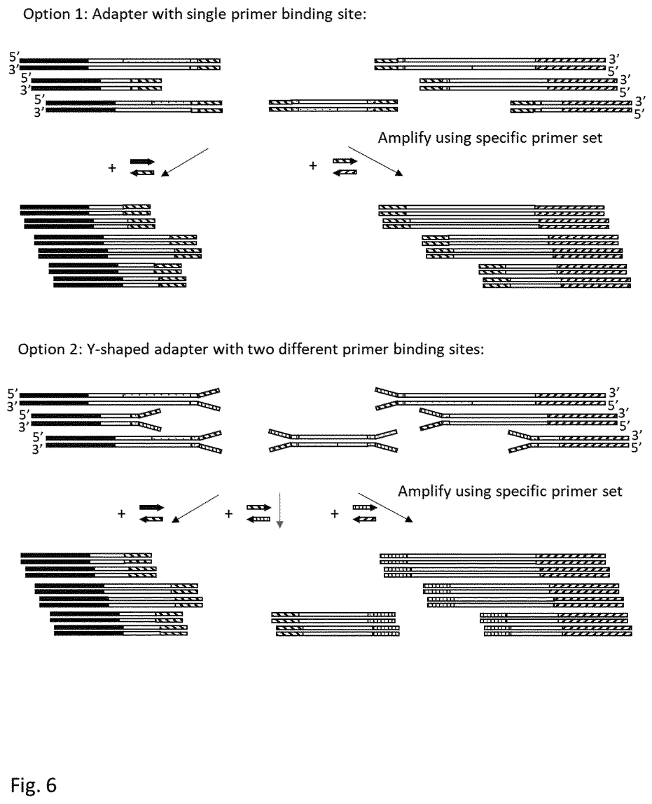

Optionally, the sequence library can be amplified before sequencing. Through the design of the adaptor, specific parts of the nucleic acid fragments can be amplified. FIG. 6 depicts multiple different adaptor designs.

In one embodiment (option 1), an adaptor with a single primer binding sequence (specific primer binding site 3; depicted with downward diagonal stripes) is ligated to the nucleic acid fragments.

After ligation, the library fragments containing the 5′ end of the original fragment can be specifically amplified using primers specific to primer binding sequence 2 and 3.

Library fragments containing the 3′ end of the original fragment can be specifically amplified using primers specific to primer binding sequence 1 and 3.

The intermediate fragments will not efficiently amplify, as fragments with the same primer binding sequences (primer binding sequence 3) will form intramolecular hairpins, which prevent the binding of primers to the primer binding sits.

In another embodiment (option 2), a Y-shaped adaptor with two different primer binding sequences (specific primer binding site 3; depicted with downward diagonal stripes, and specific primer binding site 4; depicted with vertical stripes) is ligated to the nucleic acid fragments.

After ligation, the library fragments containing the 5′ end of the original fragment can be specifically amplified using primers specific to primer binding sequence 2 and 3.

Library fragments containing the 3′ end of the original fragment can be specifically amplified using primers specific to primer binding sequence 1 and 4.

The intermediate fragments can be amplified using primers specific to primer binding sequences 3 and 4.

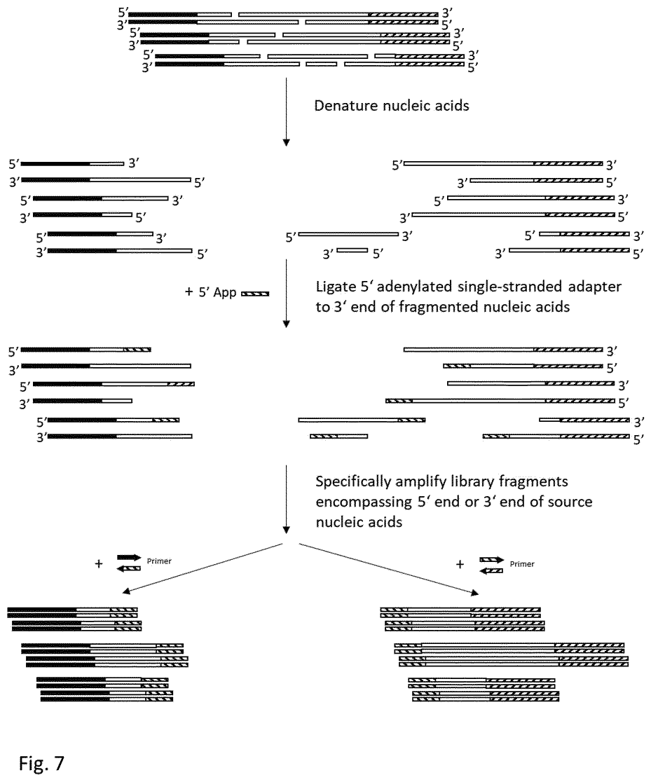

In another preferred embodiment depicted in FIG. 7, the nucleic acid fragments are first denatured (e.g. using heat or by increasing the pH): thereby, the nucleic acid fragments become single-stranded.

Next, a single-stranded oligonucleotide containing a specific primer binding site (adaptor 3 with primer sequence 3, depicted with downward diagonal stripes) is ligated to the 5′ end of the single stranded nucleic acid fragments.

In the embodiment depicted in FIG. 7, the oligonucleotide has a 5′ adenylation modification at the 5′ end (5′ App). The ligation reaction is catalysed using the Thermostable 5′ App DNA/RNA Ligase from New England Biolabs (Cat. No M0319, New England Biolabs, Ipswich, MA, USA) or an equivalent enzyme.

The resulting nucleic acid library can either be sequenced directly or amplified using specific primer sets.

By the choice of the amplification primers, it is possible to amplify a subset of the nucleic acid library: primer sequence 1 (depicted with upward diagonal stripes) and primer sequence 3 (downward diagonal stripes) for the amplification of the fragments containing the 3′ end of the original cDNA, and primer sequence 2 (solid) and primer sequence 3 for the amplification of fragments containing the 5′ end, respectively.

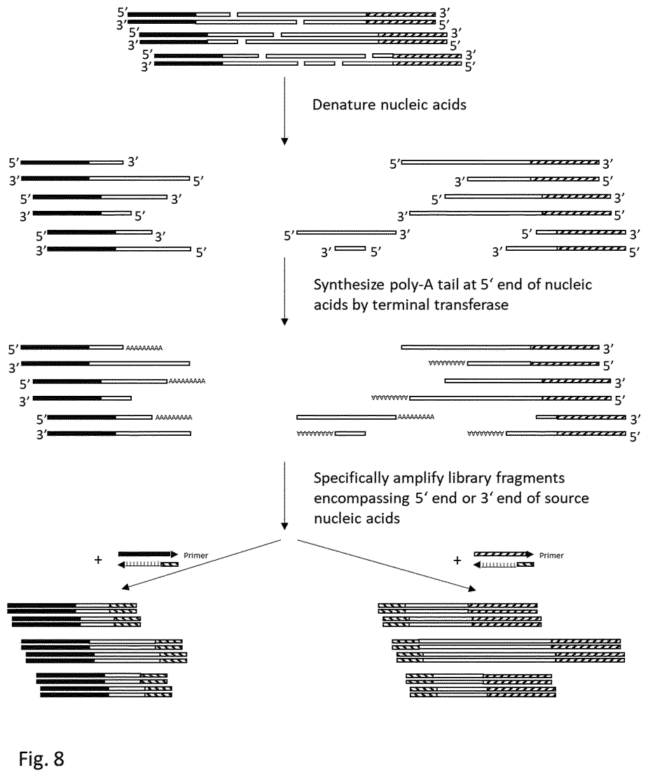

In a third preferred embodiment depicted in FIG. 8, the nucleic acid fragments are first denatured (e.g. using heat or by increasing the pH): thereby, the nucleic acid fragments become single-stranded.

Next, the single-stranded nucleic acid fragments are incubated with terminal transferase and a single oligonucleotide, thereby creating a mononucleotide tail at the 3′ end of the nucleic acid fragments.

In the embodiment shown in FIG. 8, the nucleotide is ATP, resulting in a poly-A tail at the 3′ end of the nucleic acid fragments.

In the next step, the fragments containing the 5′ end of the original fragment can be amplified by a specific primer with a poly-T stretch at the 3′ end of the primer (which binds to the poly-A tail of the library) and a primer specific for sequence 2 [depicted in solid]; the fragments containing the 3′ end of the original fragment can be amplified using the same poly-T stretch containing primer and a primer specific for sequence 1 [upward diagonal stripes]).

### REFERENCES

Melamede R J, Hatahet Z, Kow Y W, Ide H, Wallace S S. Isolation and characterization of endonuclease VIII from Escherichia coli. Biochemistry. 1994 Feb. 8; 33(5):1255-64. doi: 10.1021/bi00171a028. PMID: 8110759.
Jiang D, Hatahet Z, Melamede R J, Kow Y W, Wallace S S. Characterization of Escherichia coli endonuclease VIII. J Biol Chem. 1997 Dec. 19; 272(51):32230-9. doi: 10.1074/jbc.272.51.32230. PMID: 9405426.
Chenchik A., Zhu, Y. Y., Diatchenko, L., Li, R., Hill, J. and Siebert, P. D. (1998) Generation and use of high-quality cDNA form small amounts of total RNA by SMART PCR. In Siebert, P. and Larrick, J. (eds), Gene Cloning and Analysis by RT-PCR. Biotechniques Books, Natick, MA, pp. 305-319.
Silander K, Saarela J. Whole genome amplification with Phi29 DNA polymerase to enable genetic or genomic analysis of samples of low DNA yield. Methods Mol Biol. 2008; 439:1-18. doi: 10.1007/978-1-59745-188-8_1 PMID: 18370092.

### EXAMPLES

### Example 1: The Fragment Size can be Adjusted by the Ratio Between dUTP and dTTP During Amplification

We first assessed whether the fragment size can be adjusted by the ratio between dUTP and dTTP in a polymerase chain reaction. As model system we chose amplified cDNA generated with the template switching approach shown in FIG. 2 (generated using the Chromium Next GEM Single Cell V(D)J Reagent Kits v1.1, 10× Genomics, Pleasanton, CA, USA).

In order to statistically incorporate dUTP nucleotides, we re-amplified the cDNA for 10 cycles using the Q5U Hot Start High-Fidelity DNA Polymerase (Cat. No. M0493, New England Biolabs, Ipswich, MA, USA) according to the manufacturer's protocol. Four different relative amounts of dUTP were added to the reaction together with a no dUTP control (the percentage of dUTP refers to the fraction of dTTP replaced by dUTP in the reaction setup): condition 1: 20% dUTP, 80% dTTP; condition 2: 4% dUTP, 96% dTTP; condition 3: 0.8% dUTP, 99.2% dTTP; Condition 4: 0.16% dUTP, 99.84% dTTP; Condition 5: dTTP only.

After the amplification, an aliquot of the samples was treated with the Thermoliable USER II Enzyme (Cat. No. M5508, New England Biolabs, Ipswich, MA, USA) at 37° C. for 15 minutes followed (“USER treatment”) by a heat inactivation step at 65° C. for 10 minutes. Samples were purified using 0.8× SPRIselect beads (Cat. No. B23317, Beckman Coulter, Brea, CA, USA) and analyzed on an Agilent 4200 TapeStation System using D5000 or High Sensitivity D5000 Screen Tapes (Cat. No. 5067-5588 and 5067-5592, Agilent, Santa Clara, CA, USA).

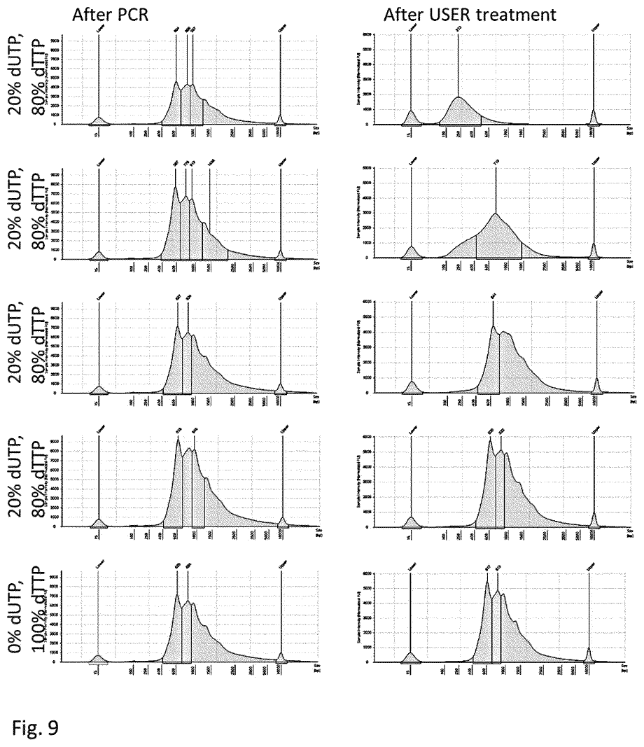

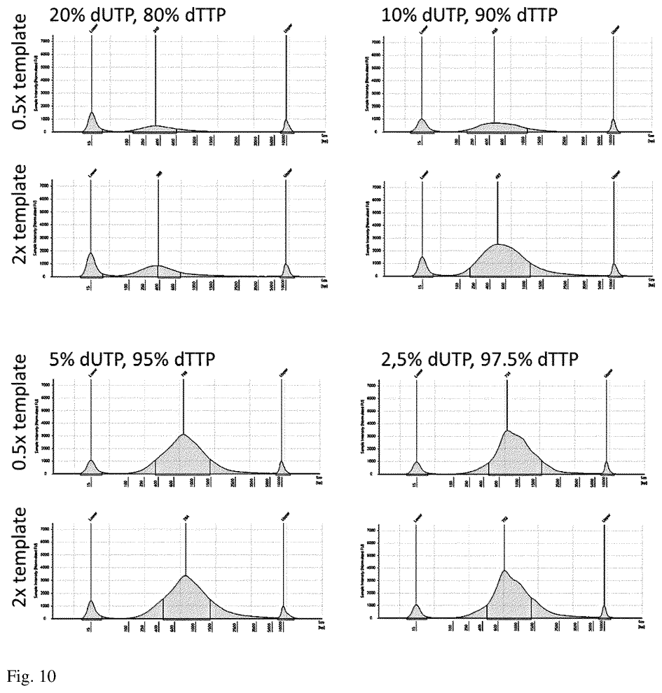

As shown in FIG. 9, the presence of dUTP at different relative fractions did not impact the size distribution of the sample after amplification (left column); after USER treatment, the amplification products were fragmented, and the fragment size was inversely proportional to the relative fraction of dUTP (right column): the larger the relative fraction of dUTP, the smaller the statistical fragment size.

### Example 2: The Fragment Size is Independent of the Template Input Amount

We next assessed whether the fragment size after USER treatment is dependent on the number of molecules used as input for the amplification reaction.

Two different input amounts were used for the initial amplification (0.5× template: 1 pg/μl; and 2× template: 4 pg/μl). Amplification and USER II treatment were conducted as described in example 1 with the exception that different relative amounts of dUTP were used: condition 1: 20% dUTP, 80% dTTP; condition 2: 10% dUTP, 90% dTTP; condition 3: 5% dUTP, 95% dTTP; condition 4: 2.5% dUTP, 97.5% dTTP.

FIG. 10 shows the results for the two different template concentrations after USER treatment: for all dUTP concentrations, the fragment distribution was very similar independent of the template concentration. This proves that the proposed method has the very unique feature that the statistical size of nucleic acid fragment does not depend on the input amount. Instead, the fragment size can be fine-tuned by adjusting the relative abundance of dUTP in an amplification reaction. This is a very unique property which facilitates workflows that do not depend on accurate quantification of the starting material or intermediate products.

### Example 3: Next Generation Sequencing Libraries Generated with dUTP/USER Enzyme Fragmented Nucleic Acids

In this example we amplified re-amplified the same template used in example 1 and 2 in 25 μl reactions (template concentration: 1 pg/μl). Amplification and USER treatment were conducted as described in example 1, and the relative amounts of dUTP were identical to experiment 2: condition 1: 20% dUTP, 80% dTTP; condition 2: 10% dUTP, 90% dTTP; condition 3: 5% dUTP, 95% dTTP; Condition 4: 2.5% dUTP, 97.5% dTTP.

10 out of the 25+1 μl were subjected to a end repair and A-tailing reaction by using the NEBNext® Ultra™ II End Repair/dA-Tailing Module (Cat. No. E7546, New England Biolabs, Ipswich, MA, USA) following the manufacturers instruction (at half scale), followed by the ligation of the 10× genomics Adaptor Mix (PN 220026, 10× Genomics, Pleasanton, CA, USA) using the NEBNext® Ultra™ II Ligation Module (Cat. No. E7595, New England Biolabs, Ipswich, MA, USA; also at half scale).

The remaining 16 μl of the amplification/Thermoliable USER II reaction were purified using 1× SPRIselect beads (Cat. No. B23317, Beckman Coulter, Brea, CA, USA). The samples were eluted in 25 μl elution buffer and subjected to the same procedure of end repair and A-tailing, and adaptor ligation as described for the non-purified counterpart.

Two μl of each pair of samples were subjected to 10 cycles of sample index PCR using the reagents taken from the Chromium Single Cell 5′ Library Construction Kit (PN-1000020, 10× Genomics, Pleasanton, CA, USA). Additionally, 10 μl of the ligation product derived from the sample already purified after the amplification/USER II step was also purified using 1× SPRIselect beads (Cat. No. B23317, Beckman Coulter, Brea, CA, USA) and subjected to the same sample index PCR protocol.

All samples were finally purified using 0.8× SPRIselect beads (Cat. No. B23317, Beckman Coulter, Brea, CA, USA).

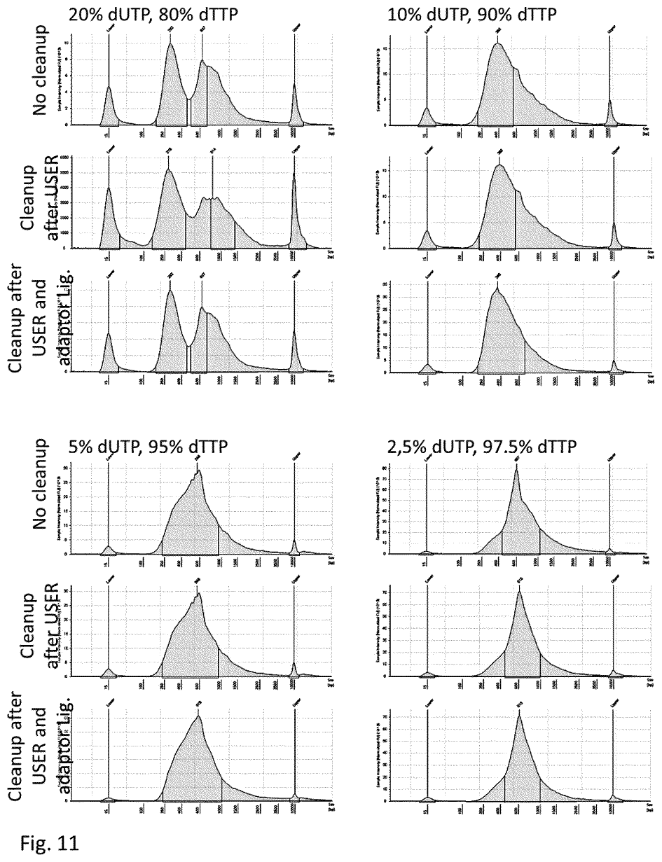

The result summarized in FIG. 11 proves that libraries can be generated with nucleic acid fragments generated with the proposed method, and that the library size is inversely proportional to the relative fraction of dUTP in the initial PCR reaction.

FIG. 11 additionally shows for all three procedures:
No purification after amplification/USER II treatment, no purification after ligation
Purification after amplification/USER II treatment, no purification after ligation
Purification after amplification/USER II treatment, purification after ligation gave rise to libraries of similar size distribution.

This observation is of great importance, as it exemplifies an additional unexpected advantage of the proposed method: During the different workflow steps, little undesired artifacts are being generated that compete with the amplification of the final library (sample index PCR), therefore the majority of amplicons generated is specific. Because of this, only one single purification (cleanup) is required to deplete fragments that are too small.

In contrast, methods of the art like the Chromium Single Cell 5′ Library Construction Kit (PN-1000020, 10× Genomics, Pleasanton, CA, USA) require a total of three cleanup steps and one size selection step, which are time consuming and lead to challenges when automating an next generation sequencing workflow.

### Example 4: Targeted Enrichment of Specific mRNA/cDNA Molecules Followed by NGS Sequencing (Targeted RNA-Seq)

To assess the applicability of the method in RNA-Seq applications requiring fragmentation of the amplified fragment after target enrichment, we used target enrichment primers provided in the Chromium Single Cell V(D)J Enrichment Kit, Human T Cell (PN-1000005, 10× Genomics, Pleasanton, CA, USA). The input cDNA used in this evaluation was previously generated using the Chromium Next GEM Single Cell 5′ Library & Gel Bead kit v1.1 (PN-100165, 10× Genomics, Pleasanton, CA, USA).

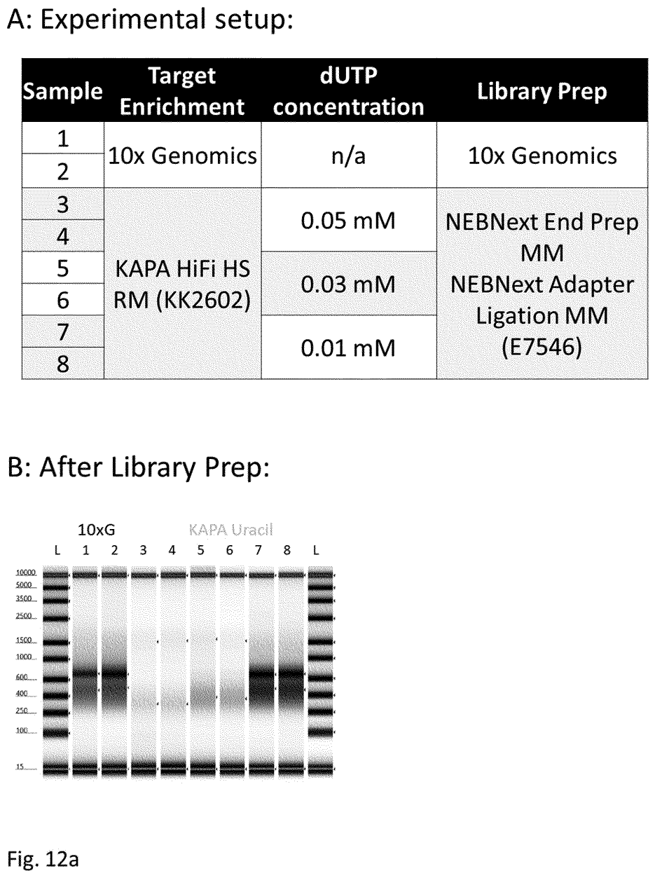

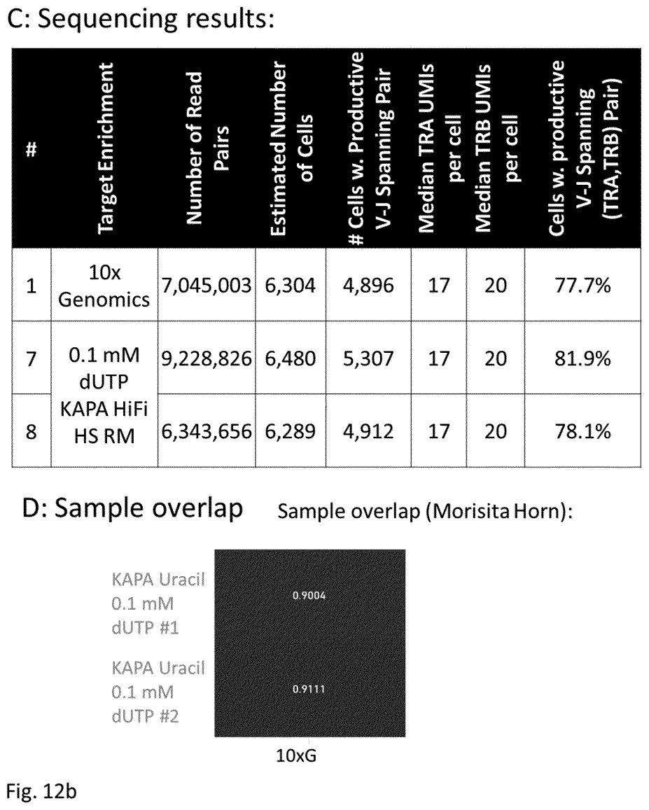

As a control, we generated a library using the respective 10× Genomics kit following the instructions provided in the 10× Genomics user guide CG000208 Rev E. The sample is depicted as ‘10×G’ or ‘10× Genomics’ in FIG. 12.

The Target Enrichment PCRs (Step 4 in the 10× Genomics user guide CG000208 Rev E) was conducted using the using the KAPA HiFi HS Uracil+RM (KK2801, Roche Diagnostics, Rotkreuz, Switzerland) using three different amounts of dUTP added to the unknown dTTP concentration in the reaction mix (final dUTP concentration: 0.05 mM, 0.03 mM and 0.01 mM, see FIG. 12A).

After Target Enrichment 1, samples were subjected to a cleanup step using SPRIselect beads (Cat. No. B23318, Beckman Coulter, Pasadena, CA, USA) according to the instructions provided in step 4.2 in the 10× Genomics user guide CG000208 Rev E.

After Target Enrichment 2, samples were subjected to a double-sided size selection according to the instructions provided in step 4.4 in the 10× Genomics user guide CG000208 Rev E followed by treatment with the USER II enzyme.

Subsequently, samples were subjected to an end repair and adapter ligation reaction as described in example 3 followed by a post ligation cleanup according to the instructions provided in step 5.3 in the 10× Genomics user guide CG000208 Rev E.

After adapter ligations, sample indices were introduced using the Single Index Kit T Set A, 96 rxns (PN-1000213, 10× Genomics, Pleasanton, CA, USA) following the instructions provided in the 10× Genomics user guide CG000208 Rev E.

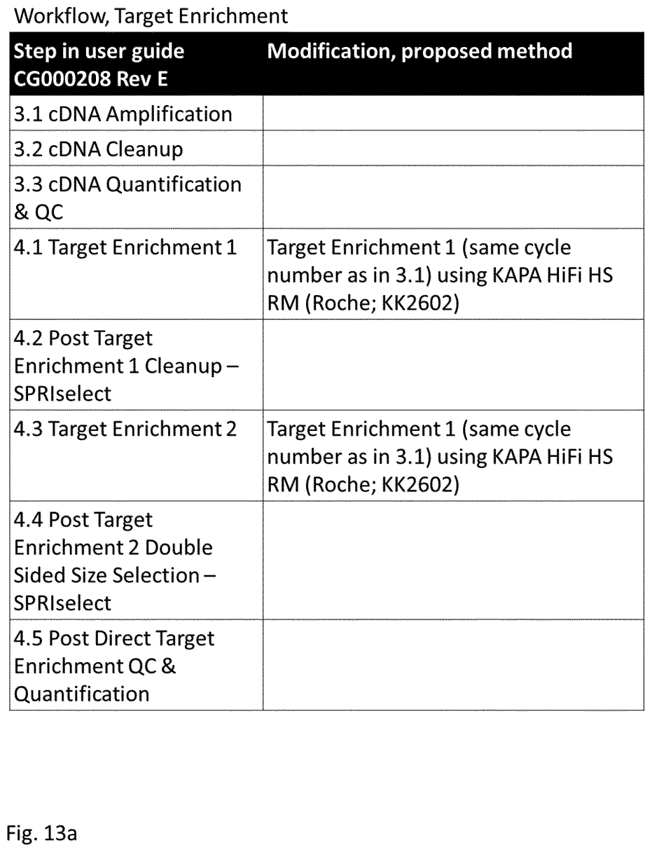

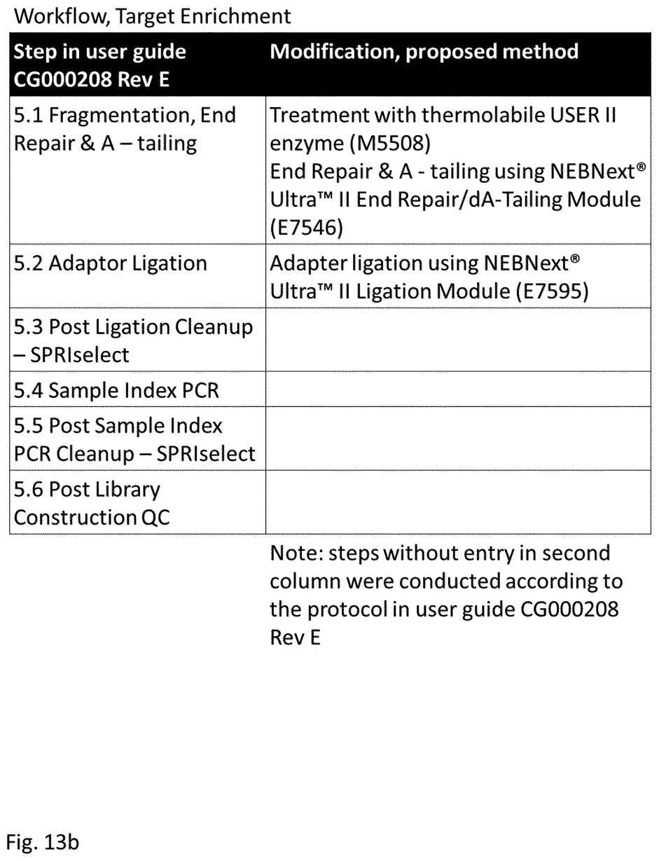

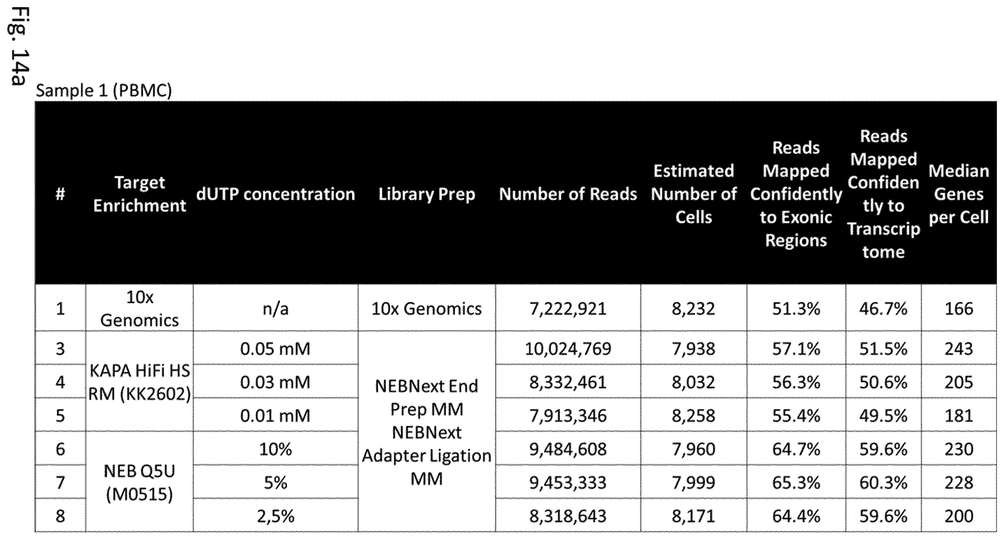

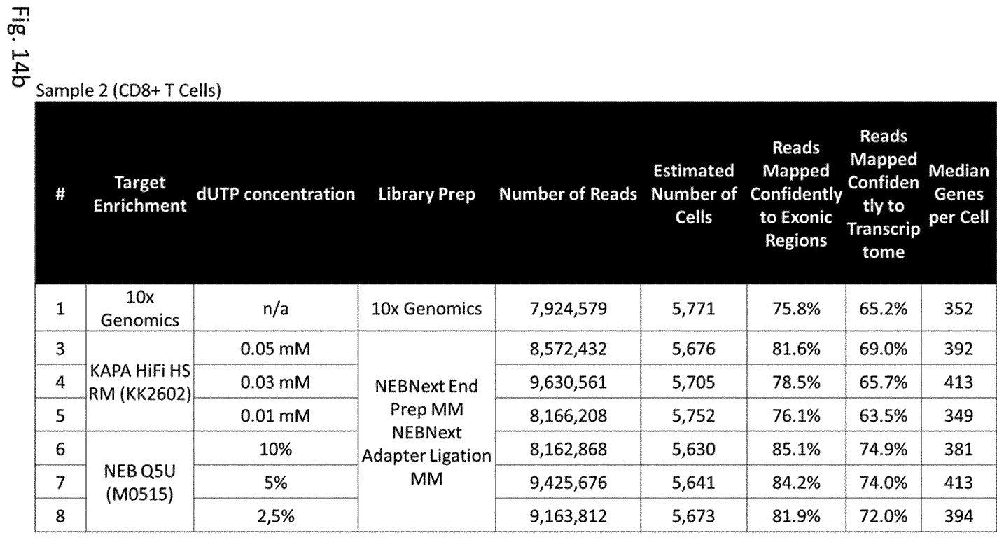

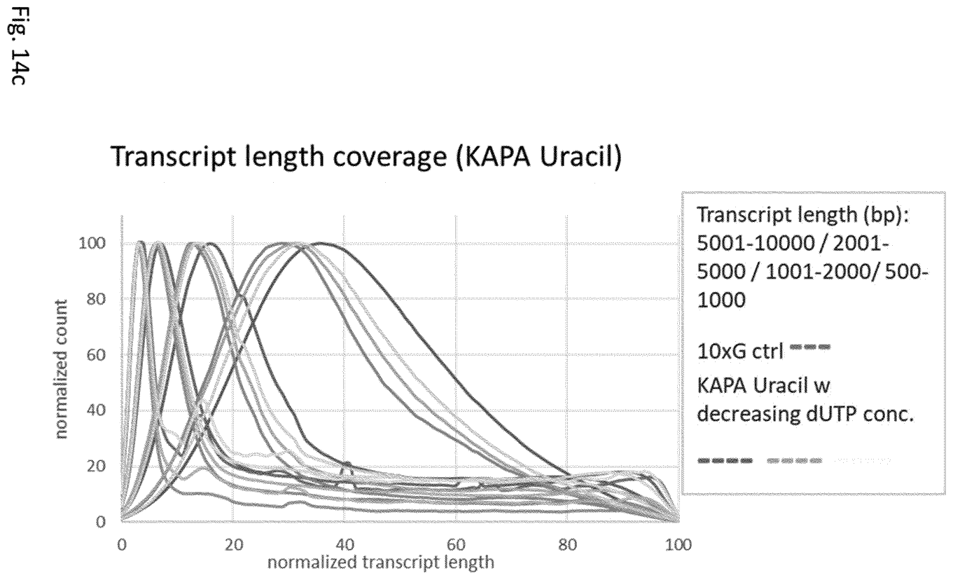

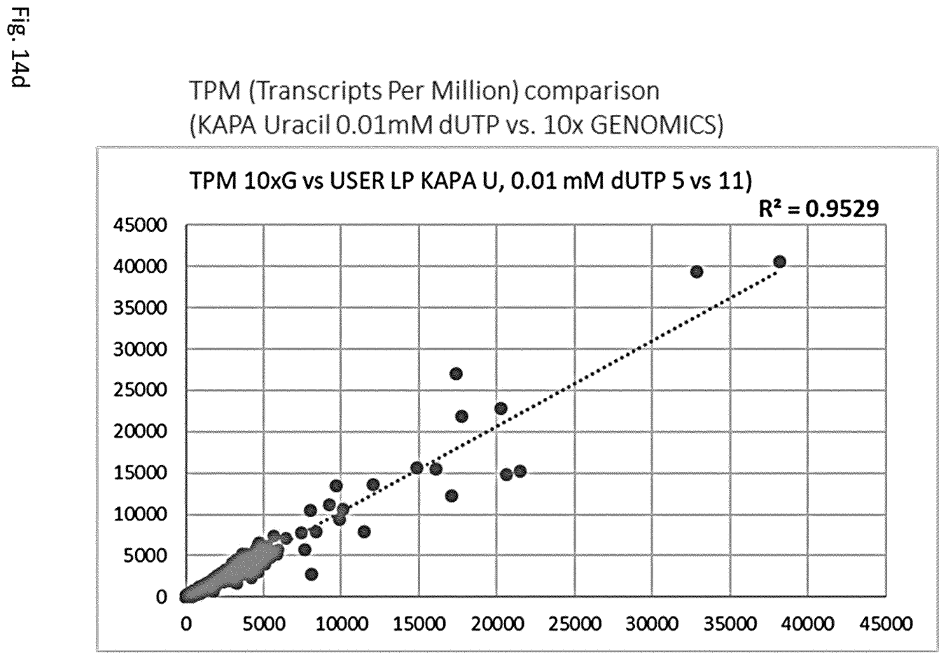

FIG. 13 summarizes the differences between the protocol used for evaluating the proposed method and the protocol proposed by 10× Genomics (user guide CG000208 Rev E).

The final libraries are shown in FIG. 12 B. The samples generated using 0.05 and 0.03 mM dUTP in the target enrichment reactions were over-fragmented (majority of fragments was below cutoff of final size selection step). The samples using 0.01 mM dUTP in the target enrichment exhibited a very nice library distribution in the desired size range of 250 to 500 bp. Surprisingly, the size distribution of the obtained libraries was even better than the size distribution of the 10× control.

Two libraries for the 0.1 mM dUTP sample and one library for a 10× Genomics control were sequenced on an Illumina NextSeq (NextSeq 500/550 Mid Output Kit v2.5 (150 Cycles), Cat No. 20024904, Illumina, San Diego, California, USA) and analyzed using the 10× Genomics Cell Ranger software (Version 6.0.1).

The sequencing results obtained for the 0.1 mM dUTP libraries confirm that libraries were more evenly fragmented as both samples showed a higher number of cells with productive V-J spanning pair for both T Cell Receptor sequences compared to the 10× genomics control (FIG. 12 C).

### Example 5: Gene Expression Analysis Using NGS Sequencing (RNA-Seq)

We also assessed the proposed method in NGS-based expression profiling. Towards this goal, we used cDNA generated from two different cDNA samples derived from human peripheral blood mononuclear cells (PB MCs) or CD8 positive human T cells (cDNA was generated using the Chromium Next GEM Single Cell 5′ Library & Gel Bead kit v1.1, PN-100165, 10× Genomics, Pleasanton, CA, USA).

For this evaluation, two different polymerases capable of incorporating dUTP nucleotides during PCR were used for the cDNA amplification step: The KAPA HiFi HS Uracil+RM (KK2801, Roche Diagnostics, Rotkreuz, Switzerland) and the NEB Q5U polymerase (M0515, New England Biolabs, Ipswich, MA, USA); for each polymerase, three different concentrations of dUTP were used (see FIG. 14).

The same two cDNA samples were also processed using the amplification enzyme provided by 10× Genomics (Chromium Single Cell 5′ Library Construction Kit, 16 rxns, PN-1000020, 10× Genomics, Pleasanton, CA, USA). Those control libraries are depicted as ‘10× Genomics’ in FIG. 14.

After cDNA amplification, samples were subjected to a size selection according to the instructions in step 3.2 in the 10× Genomics user guide CG000208 Rev E followed by a treatment with the USER II enzyme.

Subsequently, samples were subjected to an end repair and adapter ligation reaction as described in example 3. followed by a post ligation cleanup according to the instructions in step 6.4 in the 10× Genomics user guide CG000208 Rev E.

After adapter ligations, sample indices were introduced using the Single Index Kit T Set A, 96 rxns (PN-1000213, 10× Genomics, Pleasanton, CA, USA) following the instructions provided in the 10× Genomics user guide CG000208 Rev E.

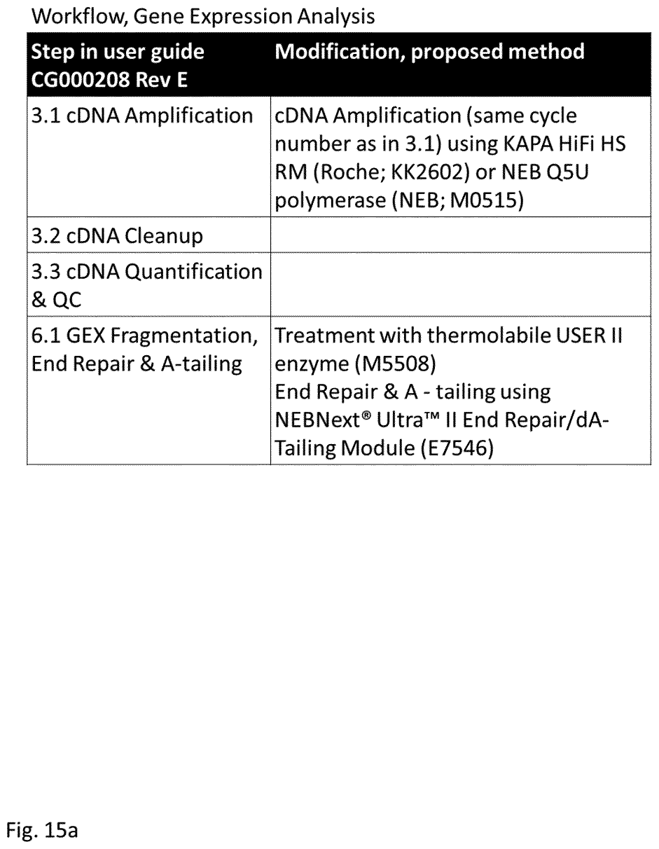

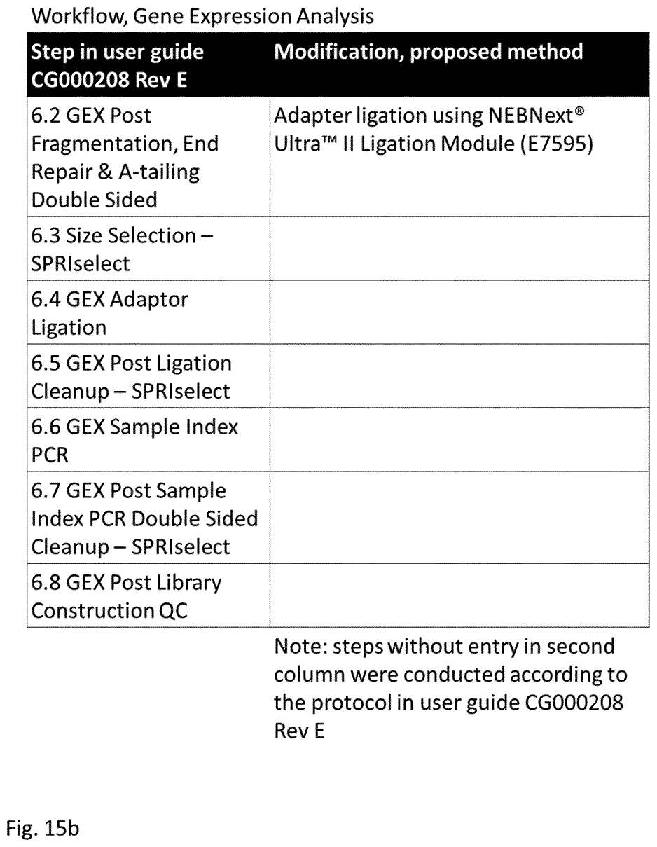

FIG. 15 summarizes the differences between the protocol used for evaluating the proposed method and the protocol proposed by 10× Genomics (user guide CG000208 Rev E).

Libraries were sequenced on an Illumina. NextSeq (NextSeq 500/550 Mid Output Kit v2.5 (150 Cycles), Cat No. 20024904, Illumina, San Diego, California, USA) and analyzed using the 10× Genomics Cell Ranger software.

All libraries generated with the proposed method displayed a higher number of reads mapped to exonic regions and the transcriptome (one outlier) (FIG. 14, top table), therefore clearly showing that the proposed method is more specific mRNA compared to the 10× Genomics workflow.

As expected, the method also allows to fine-tune the region of a transcript sequenced by adjusting the dUTP concentration during cDNA amplification (FIG. 14, bottom left; representative example of one library).

The method of the invention did not have any significant influence on the gene expression analysis (FIG. 14, bottom right; comparison of transcripts per million [tpm] for the whole transcriptome; representative example of one library is shown), indicating that the method does not lead to an observable bias in gene expression studies using human tissue or cells.

### Example 6: Omission of Cleanup/Size Selection Steps During Library Preparation for Targeted RNA-Seq

Based upon the unexpected observations in Example 3 we systematically evaluated which of the cleanup steps or size selection steps can be omitted when using the proposed method.

For this, libraries were generated using the protocol described in Example 4 (0.1 mM dUTP, KAPA HiFi HS Uracil+RM, KK2801, Roche Diagnostics, Rotkreuz, Switzerland).

We conducted two separate experiments (using cDNA generated from two different samples) and omitted one or two of the different size selection and/or cleanup steps in the protocol.

Libraries were sequenced on an Illumina NextSeq (NextSeq 500/550 Mid Output Kit v2.5 (150 Cycles), Cat No. 20024904, Illumina, San Diego, California, USA) and analyzed using the 10× Genomics Cell Ranger software.

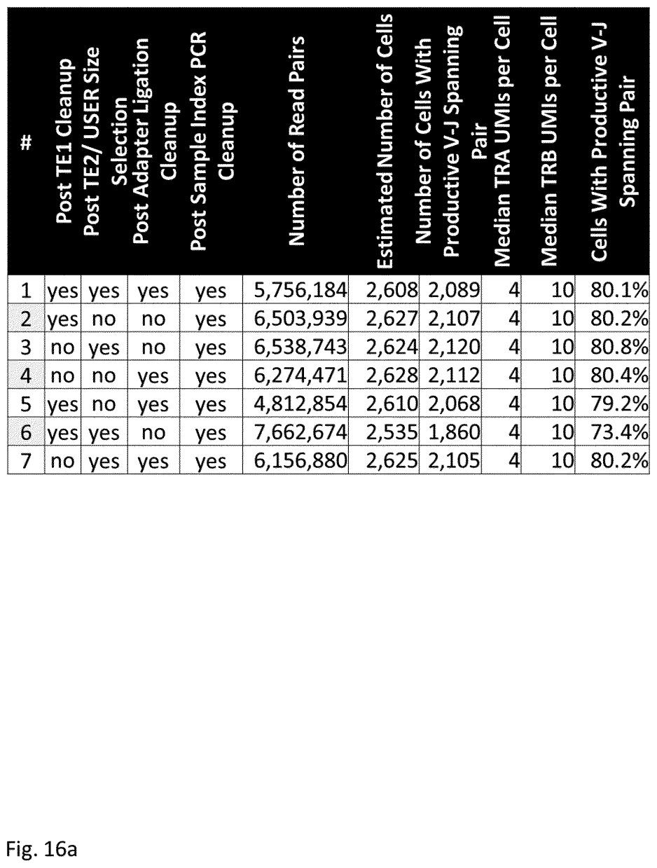

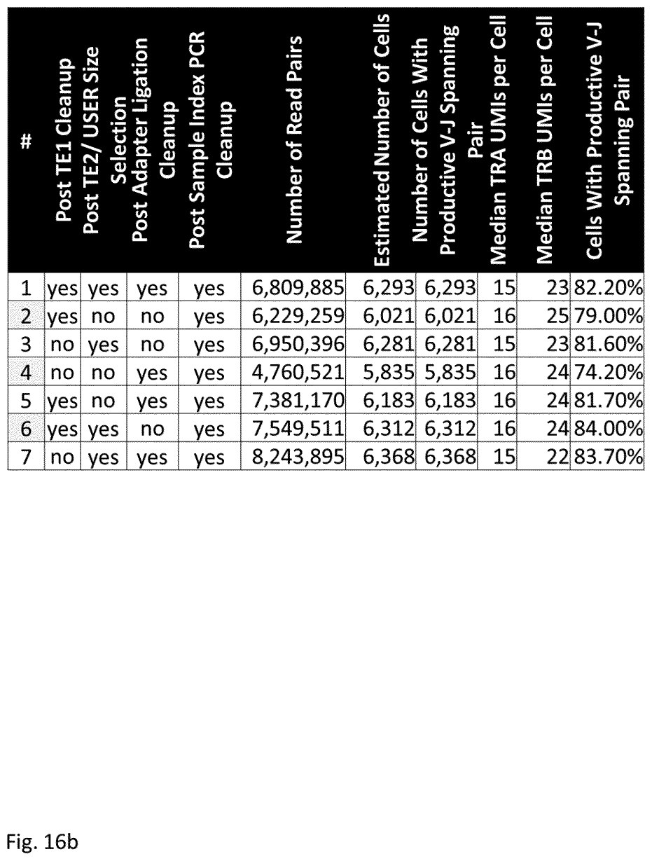

Surprisingly, we did not see any sharp decrease in performance even when omitting two of the cleanup and/or size selection steps (see FIG. 16; note that some conditions showed a slightly lower performance, but this was caused due a difference in input reads, which impacts the 10× genomics algorithm determining the number of cells).

This is in sharp contrast to methods using unspecific fragmentation (like DNase-mediated fragmentation) or tagmentation (e.g. using transposase) where these cleanup steps are crucial.

This observation again emphasizes the great advantage of the method of the invention, both for manual protocols and for automation.

### Example 7: Proposed Method Leads to Comparable Results when Using Different Amounts of Input DNA Before Fragmentation (Gene Expression Analysis)

Using the RNA-Seq protocol from example 5 (FIG. 11), we next evaluated whether the method of the invention is insensitive to the amount used for fragmentation.

Libraries were sequenced on an Illumina NextSeq (NextSeq 500/550 Mid Output Kit v2.5 (150 Cycles), Cat No. 20024904, Illumina, San Diego, California, USA) and analyzed using the 10× Genomics Cell Ranger software.

FIG. 16 clearly shows that the input amount is not critical as libraries spanning an input window of 5 ng up to 200 ng gave rise to very comparable results.

The input amount also did not have any significant influence on the gene expression analysis as a pairwise analysis of all libraries generated with the proposed method had a high correlation (R-square of over 0.99) (FIG. 15, bottom right; comparison of transcripts per million [tpm] for the whole transcriptome; comparison with representative example for each input amount is shown), indicating that gene expression analysis using the proposed method is independent of the input amount before fragmentation.

This is in sharp contrast to methods using unspecific fragmentation (like DNase-mediated fragmentation) or tagmentation (e.g. using transposase) which require tight control of the input amount.

### Example 8: Proposed Method Leads to Comparable Results when Using Different Amounts of Input DNA Before Fragmentation (Targeted RNA-Seq)

We also evaluated the impact of the input amount for the proposed method using the protocol introduced in example 4/FIG. 13.

For all libraries, target enrichment 1 and 2 were conducted using the KAPA HiFi HS Uracil+RM (KK2801, Roche Diagnostics, Rotkreuz, Switzerland) with additional 0.1 mM dUTP.

Two independent sets of experiments were conducted, a first using the target enrichment primers taken from the Chromium Single Cell V(D)J Enrichment Kit, Human T Cell, 96 rxns (PN-1000005, 10× Genomics, Pleasanton, CA, USA) and a second set using the target enrichment primers taken from the Chromium Single Cell V(D)J Enrichment Kit, Human B Cell, 96 rxns (PN-100001610× Genomics, Pleasanton, CA, USA).

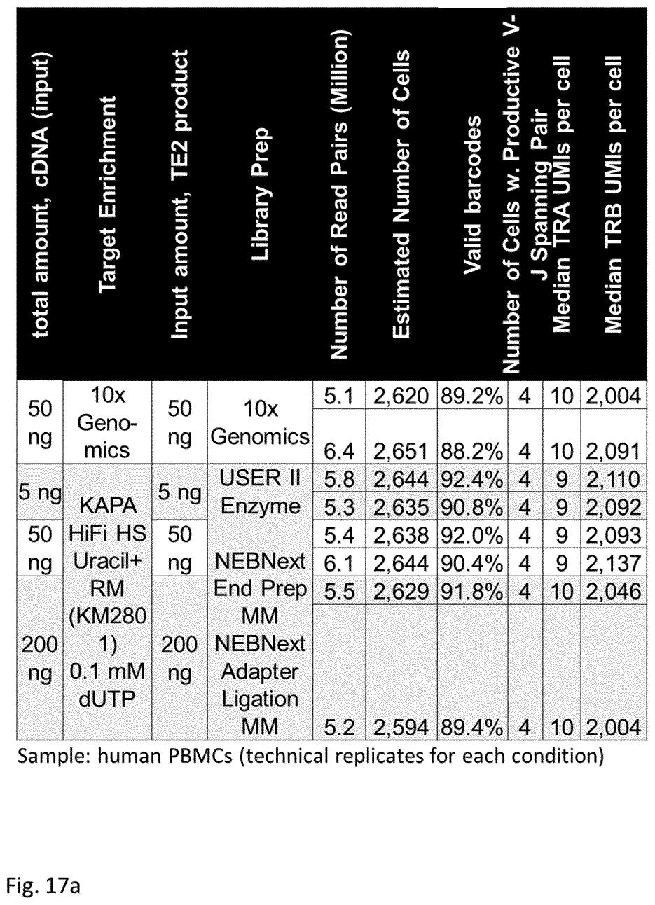

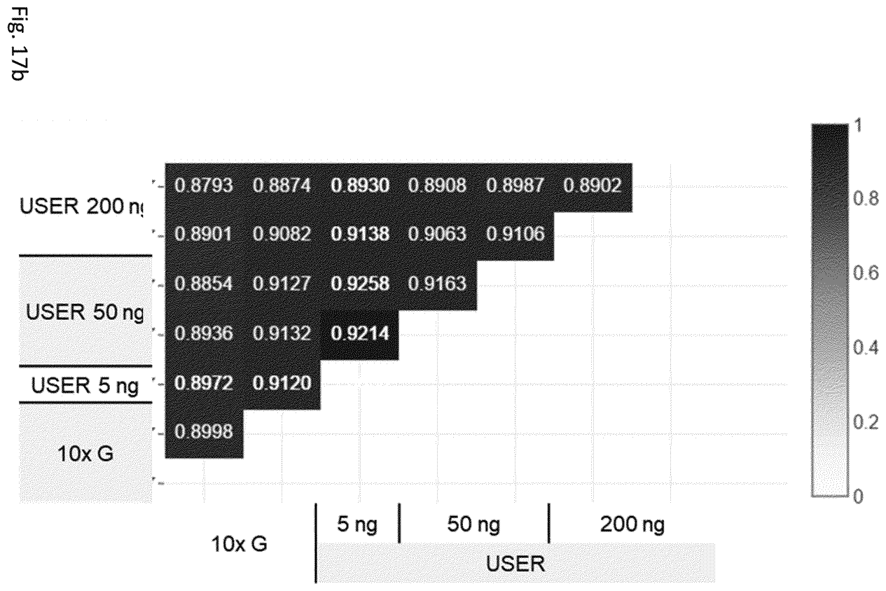

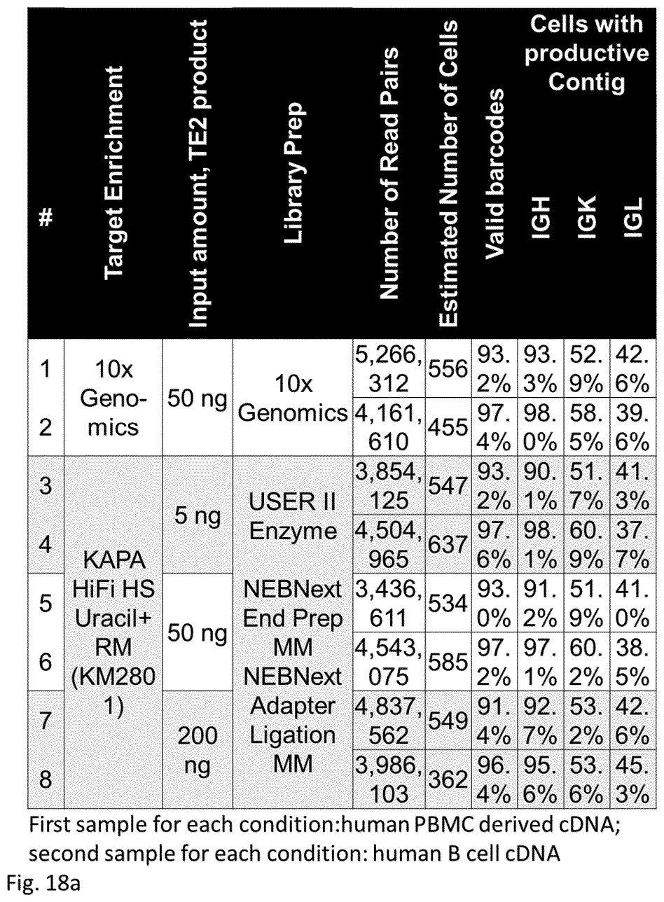

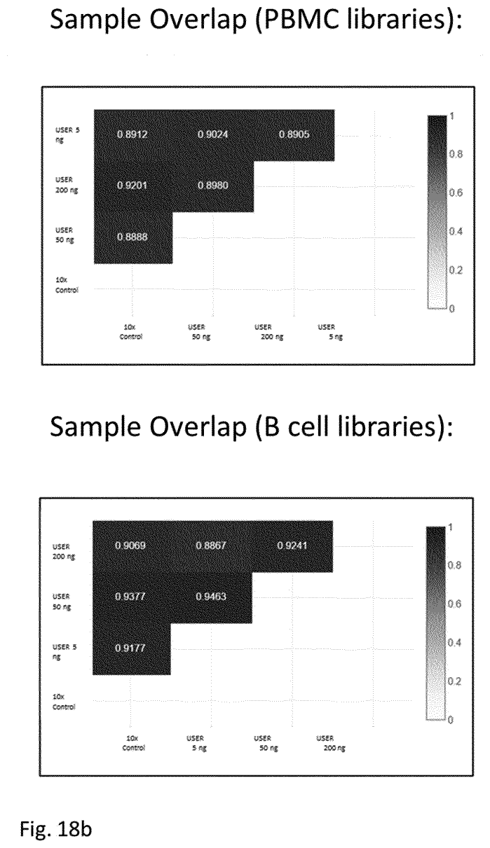

In each set of experiments a total of two libraries were generated for each condition (see FIGS. 17 and 18, top left).

Libraries were sequenced on an Illumina NextSeq (NextSeq 500/550 Mid Output Kit v2.5 (150 Cycles), Cat No. 20024904, Illumina, San Diego, California, USA) and analyzed using the 10× Genomics Cell Ranger software.

As expected, we did not see any markable differences for the libraries that were generated with the different amount of target enrichment product for the fragmentation step using the USER II enzyme.

This indicates that the proposed method leads to an even coverage of the target of interest independent of the input amount.

This is in sharp contrast to methods using unspecific fragmentation (like DNase-mediated fragmentation) or tagmentation (e.g. using transposase) which require tight control of the input amount.

1. A method for obtaining a nucleic acid library of a sample comprising polynucleotides comprising the steps
a. multiplying the polynucleotides by a polymerase
b. fragmenting the multiplied polynucleotides by creating nicks
c. coupling an oligonucleotide sequence to the nicks to create the target library
wherein step a) is performed by providing A, T, G, C and U nucleotides wherein the molar ratio of T and U is between 200:1 and 5:1 and step b) is performed by excision of the U nucleotides. characterized in that after step b), the nicks are provided with a polymerase exhibiting 5′→3′ exonuclease activity, thereby filling in the 3′ recessing ends and removing the 5′ overhangs of the nicks.

2. The method according to claim 1 characterized in that the A, T, G, C and U nucleotides are provided as Adenosine 5′-Triphosphate (ATP), 2′-Deoxyadenosine 5′-Triphosphate (dATP), Thymidine 5′-Triphosphate (TTP), 2′-Deoxythymidine 5′-Triphosphate (dTTP), Guanosine 5′-Triphosphate (GTP), 2′-Deoxyguanosine 5′-Triphosphate (dGTP), Cytidine 5′-Triphosphate (CTP) and 2′-Deoxycytidine 5′-Triphosphate (dCTP), 2″-Deoxyuridine, 5″-Triphosphate (dUTP) or Uridine-5′-triphosphate (UTP) or a derivate thereof.

3. The method according to claim 1 characterized in that at least one of the steps a) b) or c) is performed without purification of the obtained product.

4. The method according to claim 1 characterized in that step c) is performed by providing a ligase.

5. The method according to claim 1 characterized in that after step b), the nicks are denaturated into single strand nicks and the single strand nicks are provided with a ligase which couples oligonucleotide sequences to the 3′ end of the single strand nicks.

6. The method according to claim 1 characterized in that after step b), the nicks are denaturated into single strand nicks and the single strand nicks are provided with a terminal transferase which couples homonucleotides comprising 2 to 20 nucleotides as oligonucleotide sequences to the 3′ end of the single strand nicks.

7. The method according to claim 1 characterized in that the oligonucleotide sequences coupled to the nicks are primer sequences.

8. The method according to claim 1 characterized in that the polynucleotides are derived from synthetic or genomic DNA or RNA or a plurality of DNA or RNA molecules comprising 50 to 2000 nucleotides.

9. The method according to claim 1 characterized in that before step a, the polynucleotides are provided at the 3′ and 5′ ends with primer sequences for amplification.

10. The method according to claim 8 characterized in that the primer sequences are same or different than the oligonucleotide sequences.

11. The method according to claim 1 characterized in that after step c, the target library is amplified.

12. The method according to claim 1 characterized in that multiplying the polynucleotides in step a) is conducted by polymerase chain reaction.

13. The method according to claim 1 characterized in that nicks are generated by a providing one or more enzymes selected from the group consisting of DNA glycosylases, endonucleases, engineered recombinant proteins and thermolabile engineered recombinant proteins.

14. The method according to claim 1 characterized in that the nucleic acid library is sequenced.
